(19) United States
(12) Patent Application Publication
(10) Pub. No.: US 2007/0196915 A1
(43) Pub. Date: Aug. 23, 2007

(54) Cellular tissue culture systems for high-volume processing

(75) Inventors: Randall E. Tagawa, Bloomfield, CO (US); Kenneth K. Tagawa, Brighton, CO (US); George H. Tagawa, Brighton, CO (US); William A. Kluth, Broomfield, CO (US); Sarada Krishnan, Westminster, CO (US); Cindy Wieland, Highlands Ranch, CO (US)

Correspondence Address: SANTANGELO LAW OFFICES, P.C., 125 SOUTH HOWES, THIRD FLOOR, FORT COLLINS, CO, 80521, US

(73) Assignee: Tagawa Greenhouses, Inc., Brighton, CO (US)

(21) Appl. No.: 10/590,919

(22) PCT Filed: Feb. 25, 2005
(86) PCT No.: PCT/US05/05964

§ 371 Date: Aug. 25, 2006

Related U.S. Application Data
(60) Provisional application No. 60/548,847, filed on Feb. 27, 2004.
(60) Provisional application No. 60/559,981, filed on Apr. 5, 2004.

(51) Int. Cl. C12N 5/06; C12N 5/04; C12M 3/00

(52) U.S. Cl. 435/325; 435/419; 435/289.1

(57) ABSTRACT

Tissue culture medium such as porous frameworks and even open surface multidirectional porous frameworks may be used to provide uniform distribution of nourishment solutions, uniform interstitial voids as well as undistorted transport fields which may facilitate high volume yields of finished plants from cells, such as explants in a tissue culturing process. Further embodiments may include automating a tissue culturing process to reduce labor costs and increase uniformity of finished plants through tissue culture processes.

This international application claims the benefit of U.S. Provisional Application No. 60/559,981, filed Apr. 5, 2004 and U.S. Provisional Application No. 60/548,847, filed Feb. 27, 2004, hereby incorporated by reference herein.

### TECHNICAL FIELD

Generally, this invention relates to systems for tissue culture generation of plants which may increase the yield of tissue cultured plants, and may even increase the efficiency of labor in performing the tasks related to traditional tissue culture processes as well as reduce the total process time. The present invention focuses upon techniques and technology which, in turn, may result in reduced mortality of tissue cultured plants thereby perhaps even increasing a yield of finished tissue cultured plants. The present invention may reduce the number of steps used in traditional tissue culture processes possibly through the use of automated transfer methods and equipment and may provide a more effective method for delivery of plant growth hormones, nutrients and the like to the tissue culture plants. A porous framework may allow capillary action for uniform distribution of air, plant hormones and nutrients and may even maximize the development of the tissue cultured plants.

### BACKGROUND OF THE INVENTION

The use of tissue culture for plant production has been used for many years. Yet, traditional tissue culture may cause high mortality rates and high labor costs. Therefore it may be used currently only for a few high value crops such as exotic tropical plants and flowers, certain food crops and even certain commercial crops such as lumber. One advantage of tissue culture may be that it may produce an exact phenotypic and genotypic clone of the mother or stock plant that is being tissue cultured. Currently plant breeders and plant production companies may only use tissue culture to produce a very small select group of mother or stock plants which are then propagated using other less expensive methods. They may use tissue culture on those species and varieties that are difficult to or cannot be propagated using other less expensive methods. Tissue culture may be limited, therefore, to those few crops that can be sold at a premium price to recover the high costs of tissue culture.

The basic tissue culture process may include harvesting a selected small part of a growing mother or stock plant. This small part of the mother or stock plant may be surface sterilized using standard procedures known in the industry. Using sterile equipment in a sterile environmental hood that may have a positive pressure to prevent the inclusion of air borne contaminates, a small part of a mother or stock plant may be cut using a scalpel and forceps. This piece of the small part of the mother or stock plant may be called an explant. Traditionally, each step in the tissue culturing process may require manually handling of the explants which may be both labor intensive and may increase the likelihood for the introduction of disease through contamination and explant mortality. The explants may be traditionally placed on a medium containing agar and a predetermined concentration of plant growth hormones and nutrients. The cells of the explants may differentiate on this medium into root and even shoot buds based on the concentration of plant growth hormones and nutrients. This may be called Stage 1.

After a specific amount of time—which may vary from species to species and variety to variety within a species—the explants may be transferred to a new medium containing different concentrations of plant growth hormones and nutrients. On this medium the shoot and root buds may be encouraged to develop and grow. This may be called Stage 2.

After a specific amount of time—which may vary from species to species and variety to variety within a species—the explants may be transferred again to a new medium containing different concentrations of plant growth hormones and nutrients. On this medium the developed shoot and root may be encouraged to continue to grow until shoot, root and leaves may be clearly visible and the explants mature into plantlets. This may be called Stage 3.

After a specific amount of time—which may vary from species to species and variety to variety within a species—the explants may grow into plantlets and the plantlets may be transferred to a new container of various sizes containing a media (this may not be agar) in a greenhouse or other non-sterile environment to allow the plantlets to mature and become a new finished plant. This may be called Stage 4. It is well understood by those in the industry that Stage 4 may require some form of support structure to allow for the complete development of roots and shoots to maturity. Stages 1 through 3 may be conducted in the sterile environment of a laboratory using standard tissue culture equipment and techniques. Stage 4 may not need to be conducted in a laboratory but still may require technical equipment to ensure the successful maturation of the newly formed plantlet from explants. Manual grading of the explants or plantlets may occur between stages to insure that the explants or plantlets that are transferred from one stage to another are uniform in size and development. Uniformity of size and development may greatly increase yield, but manual processing may be expensive and may increase overall production costs.

Disease in plants is not acceptable. It can diminish the value of a crop by reducing the productivity of the crop through either death of the plant or poor quality finished crops. Many diseases may not be specific to a single species or variety which may allow the spread of disease from the host plant to other plants or crops. Most plants may be propagated using traditional methods which may not be automatically screened for the presence of disease. Since Sep. 11, 2001, the threat to food or other commercial crops through bio-terrorist introduction of disease may have been raised due to awareness of the vulnerability of basic food and commercial crops to contamination by disease from a host plant that may be imported or native.

The sterile medium which may be used in Stages 1 through 3 may not only encourage the transformation of the explants into a plantlet yet may also encourage the growth of any contaminates such as fungi and bacteria. Because the size of the explants may usually be very small, any fungi or bacteria or the like that may be present inside or within the explants could grow on the medium as well, indicating that disease or contamination may be present. Therefore, any explants that may survive from Stage 1 to Stage 4 could be considered to be mostly free from fungi and bacterial disease.

The present invention, in embodiments, may focus on a process using various improved support structure systems that may allow for the economic tissue culture production of plants. This may allow any plant to be economically produced using this process, not just high value crops. This may also decrease the likelihood of the introduction of disease through the traditional propagation method of using a mother plant that may have a disease that has not expressed itself. A diseased mother plant may produce hundreds of diseased plants through traditional propagation methods.

As noted, tissue culture has been used for propagation of plants for many years. There are many different concentrations of different plant growth hormones and nutrients that are used both within a species and/or variety and between species and varieties. The concentration of hormones, nutrients, and the like may vary throughout the tissue culture stages. Several methods have been published using support structure systems which may reduce labor associated with tissue culture production. These known support structures may not adequately address improving the yield of the finished tissue cultured plants through more uniform distribution of plant growth hormones and nutrients and may not allow for automation during the stages of the tissue culture process, among other reasons.

One type of support structure is noted in International Publication Number WO 87/00394 to Nippon Steel Chemical Company. This publication may describe a support structure system using ceramic fibers. The ceramic fibers may support explants in Stages 1 through 3 without the need to transfer by hand between each stage. New concentrations of plant growth hormones and nutrients may be poured, sprayed or dripped onto the ceramic fibers and the direction of the fibers may affect any capillary action of a liquid. In addition, a size of the voids between the fibers may determine the quality of the capillary action of the plant growth hormones and nutrients. Lack of uniformity of both the size of the ceramic fibers and the voids between the fibers may even result in ununiform or non-uniform distribution of plant growth hormones and nutrients.

The uniformity of distribution of plant growth hormones and nutrients may be important throughout Stages 1 through 3, and may be particularly important in Stage 1 in order to differentiate the cell structure of the explants to form into shoot and root buds. Ununiform or non-uniform distribution may result in fewer root and shoot bud formations which may decrease the yield or even the potential quality of each explant. It may even result in the death of explants possibly due to inadequate plant growth hormones or nutrients. Uneven growth may result which may cause uneven maturity periods that could even result in the need for manual grading of the explants or plantlets for quality control which is labor intensive and therefore increases labor costs.

Another problem of using ceramic fibers may be that as the fibers may need to be molded into a size and shape useful for tissue culture production. After the ceramic fibers are molded, they may have to be cut. The compression of the ceramic fibers during the cutting process may fundamentally change the voids between the fibers. A terminal or cut end of the ceramic fibers may be where the explants rest on the support structure and these ends may be sharp enough to damage or perhaps even pierce the cell structure of the explants which may reduce the explants vigor. A damaged cellular structure may increase the length of time for the explants to have cellular differentiation, development of shoot and root buds and even the maturation from an explant into a plantlet.

The ratio of a surface area of the explants that may be in contact with the ceramic fibers may be decreased because the ceramic fibers may be hard and even nonconforming to a shape of the explants. The surface area of the explants that may be in direct contact with the plant growth hormones and nutrients may not be optimal and thus may be reduced with this type of structure. Lack of contact with nutrients and the like may result in fewer root and shoot bud differentiation in Stage 1 and may result in poor yields. In Stages 2 and 3, root and shoot growth may not be uniformly encouraged possibly resulting again in increased production time, lower yields and even ununiform maturity periods which may cause increased production costs.

Because yields in traditional Stage 1 tissue culture may be as low as about 50% or less, any additional reduction of yield may greatly increase production costs perhaps even regardless of any labor savings due to fewer transfers between Stages.

During root development in Stages 2 and 3, it may be important that the ratio of air to liquid may be properly maintained so that the roots may not die from drowning. Ununiform or non-uniform voids due to irregular ceramic fibers and even compression of fibers during the cutting of the fibers into a usable shape could create voids having either too much air or too much liquid. An uneven balance of air to liquid may possibly reduce the development of roots or even possibly prevent root development into a medium. Lack of root development could increase the time during Stages 2 and 3 and may increase the mortality rate of the plantlet during Stage 4 when the plantlet may no longer be in a controlled environment of a laboratory. This may increase production costs making the process uneconomical.

Another problem with a ceramic fiber support structure may by that it may not lend itself to automation of transfer from one stage to another or perhaps even throughout the tissue culture process. In this case, the ceramic fibers may need to be unidirectional so that it could split or break along directional lines. During automation, it may be difficult to utilize equipment that can move the ceramic fibers without damaging or even splitting the ceramic fiber unit. Here, transfers between stages may require a manual process. This may increase labor costs and overall production costs.

Another support structure as described in U.S. Pat. No. 4,586,288 to Walton may include an expanded foam with a gel and a membrane. The membrane may be pierced and an explant may be placed in the pierced surface of the assembly. This piercing process may be done manually which may not consistently produce uniformity. The ununiform or non-uniform aperture of the membrane could prevent easy insertion of the explants onto the medium thereby possibly increasing the time to transfer the explants onto the medium and may increase labor costs. It may also prevent the explants' shoot development from growing upward in a natural way because the shoots may have to pass through the membrane.

The membrane may pose another problem in that it may prevent the uniform distribution of new concentrations of plant growth hormones and nutrients because the membrane may cover the medium. In order for new concentrations of plant growth hormones and/or nutrients to be applied, the old plant growth hormones and nutrients may need to be rinsed from the existing medium. This may require (due to gravity) that the new liquid be applied from the top of the support structure and rinsed downward. In this particular assembly, it may not be adequately feasible to rinse the medium in a downward motion due to the membrane. This may prevent the thorough rinsing of a previous concentration of plant growth hormone and nutrient out of the medium.

Because the membrane may be manually pierced, the piercing action could likely also pierce the medium below it. This may result in crushed or damaged medium that could prevent the uniform capillary action of the liquid medium. It could also result in different ratios of surface area of the explants to the surface area of the medium from one explant to another. This could result in uneven differentiation of root and shoot buds during Stage 1 and uneven development of those root and shoot buds during Stages 3 and 4. The plantlets may need to be graded by size in order to increase yield in Stage 4 which may result in an increase in the amount of time and labor needed earlier in the tissue culturing process. Also, the inconsistency resulting between plantlets could mean that some of the plantlets moving into Stage 4 could be immature and could possibly die. This may result in decreased yields and increased production costs due to the labor to grade, transfer and then to discard the dead plantlets.

Yet another problem with a membrane may be that because it may cover the entire surface of a medium, it may prevent any automation from occurring. Automation may require easy and complete access to a medium. A membrane could prevent extraction of the support structure by automation thereby increasing labor costs during any transferring processes. Further, a membrane may make manual transfers more difficult because of the need to cut away the membrane without damaging the developing explants and plantlets. This may increase labor costs.

Another problem with an assembly as disclosed in the Walton patent, may be that it may employ a hygroscopic gel in a medium which could attract water. A gel that may attract liquid or even water may restrict the natural capillary action of a medium. The gel may thereby possibly reduce the effectiveness of plant growth hormones and other nutrients due to ununiform or non-uniform capillary action or ununiform or non-uniform delivery of the required plant growth hormones and nutrients. This could result in slower differentiation of cells into root and shoot buds during Stage 1 and development of those root and shoot buds in subsequent Stages 2 and 3. A plant nutrient level may need to be more closely monitored due to a gel.

Before the addition of new concentrations of plant growth hormones and nutrients, the old concentrations of plant growth hormones and nutrients may need to be completely rinsed out in order to be effective. Remaining old plant growth hormones and/or nutrients combinations with new plant growth hormones and nutrients may not produce consistent cell differentiation and subsequent development of root and shoot buds. Without consistent and uniform differentiation and development of root and shoot buds, manual grading of the explants and plantlets may be necessary between each stage possibly increasing labor costs and preventing the opportunity for automation of the transfer process. Increased water availability from the hygroscopic gel may also cause increased water intake by the explant or plantlet which may increase the likelihood of vitrification (a translucent water soaked succulent appearance) which leads to mortality and reduces yields.

Other types of support structures as noted in EP 0692929B1, may suspend the explants and plantlet on a platform above a liquid medium. The platform base may have a porous material that may allow the liquid medium concentration of plant growth hormones and nutrients to pass through and come in contact with an explant or plantlet. The problem with this type of support structure may be that the amount of medium and therefore concentration of plant growth hormones and nutrients may be dependent on the porosity of the platform. As the explants and plantlets mature, they may become larger and therefore heavier and may place more downward pressure on the platform. The maturing explants and plantlets may even push more of the liquid medium through the pores of the platform. Some inventions may compensate for an increased pressure on the liquid medium below, yet there could be potential for inconsistent dispersion of the plant growth hormones and nutrients due to the increased mass of the explants and plantlets and the mechanical action of the floating platform. This may result in an uneven distribution of plant growth hormones and nutrients that could result in ununiform or non-uniform cell differentiation and development of root and shoot buds. This may lower overall yield and may result in the need for manual grading of explants or plantlets that may increase labor costs. Because the developing roots of the explants or plantlets may not be supported, it may be impossible for the process to be automated other than the movement of the entire platform to a new medium. Therefore there may be limited ability to move the developing explants and plantlets from a high density to a lower density. This may result in the need to use a lower density of explants to begin with which may use expensive laboratory or sterile environment space uneconomically. The developing explants could be manually transferred to a new platform at a lower density which may cause increased labor and may increase overall production costs.

In fact, as the present invention demonstrates, efforts such as those by Nippon Steel Chemical Company and Walton may have actually taught away from the direction of the present invention. To some degree it may even be true that the results can be considered unexpected to those skilled in the art who may have been lead to believe that solutions lie in the directions shown in the Nippon Steel Chemical Company and Walton inventions or who might have been lead to believe that the problem itself had difficulties which were to be considered inevitable. Thus, until the present invention no one had provided a porous framework system for tissue culture application which could not only be efficient but which could permit control of the growing explants throughout the entire process and achieve the yield desired without excessive labor and with a high volume production result.

### DISCLOSURE OF INVENTION

The present invention includes a variety of aspects, which may be selected in different combinations based upon the particular application or needs to be addressed. In embodiments, the invention may include improved tissue culture growth media for tissue culture of plants that may allow for the reduction of labor during Stages 1 through 4. The present invention may employ automated methods and equipment, uniform distribution of plant growth hormones, nutrients and the like, and increased yields of maturing explants and even finished plantlets in all stages. Overall the invention may allow a uniform development of tissue cultured plants.

Examples of improved support structures may include materials which can be properly sterilized, can provide uniform delivery of plant growth hormones, nutrients and the like, can result in uniform differentiation of cells and development of root and shoot buds, and can even result in increased yields.

Accordingly, one goal of the invention may be to provide uniform distribution of plant growth hormones, nutrients and the like solutions throughout a tissue culture growth media.

Another goal of the invention may be to provide adequate contact of nutrient solution and the like solutions to an explant and growing explant.

Yet another embodiment of the present invention may be to provide a system to apply and remove nourishment solutions and the like solutions to a tissue culture growth media.

Even yet, another embodiment of the present invention may be to provide uniform voids within a tissue culture growth media which may contribute to the supply of a nourishment solution to an explant and may even enhance uniform growth of a plurality of explants. It may also be a goal of the invention to provide a undistorted transport field at least near if not throughout a tissue culture growth media which may allow optimal supply of nourishment solutions and the like solutions to an explant.

Another goal may be to provide a balance of air to nourishment solution within a tissue culture growth medium. Depending on the type of plant being tissue cultured, it may be desirable to have more water, such as for tropical plants or water plants, or it may be desirable to have more air, such as for desert and drought tolerant plants.

Another goal of the invention may be to reduce labor costs through automation of the transfer of the growing explants during stages. The improved support structure systems as described later could provide uniform development of the explants and plantlets which may eliminate the need for manual grading of the explants or plantlets. This could allow for automation of the transfer between stages, such as a punch system. Automation could allow for multiple explants or plantlets to be transferred between stages which may greatly reduce labor and production expenses and increase profits. Automation methods and equipment may include processes and procedures that employ machines that may automatically apply new concentrations of plant growth hormones, nutrients and the like both during a specific stage as well as between stages.

One method of transfer (thought not necessarily the only method of transfer) may be described in International Publication Numbers WO 02/058455 and WO 02/100159 to Tagawa Greenhouses, Inc., hereby incorporated by reference. These publications may describe a process that transfers growing plants or plantlets between stages by punching the plant or plantlet downward through the bottom of a web matrix that may hold the supporting structures with the plants or plantlets. Here, these systems may have proven to be highly successful in the transfer process and could uniquely allow for the transfer of many different stages of explants or plantlets development.

Naturally further objects of the invention are disclosed throughout other areas of the specifications and claims.

### BRIEF DESCRIPTION OF THE DRAWINGS

FIGS. 1A-L shows in various embodiments, an overview of some of the steps in the tissue culture process.

FIG. 1A shows the mother or stock plant.

FIG. 1B shows the harvest of a portion of the mother or stock plant.

FIG. 1C shows the harvest of a small section of the mother or stock plant making an explant.

FIG. 1D shows a cross section close-up of an explant on a porous framework.

FIG. 1E shows a view of a web matrix of improved support structures.

FIG. 1F shows a close up of the cellular differentiation into root and shoot buds.

FIG. 1G shows a web matrix of porous framework being automatically rinsed with new nourishment solution where the old nourishment solution may be rinsed through the bottom of the web matrix.

FIG. 1H shows a close up of root and shoot development in Stage 2.

FIG. 1I shows the automated transfer of the initial web matrix of high density to a web matrix of lower density.

FIG. 1J shows a close up of root and shoot development in Stage 3.

FIG. 1K shows a transfer from Stage 3 to Stage 4 into new media.

FIG. 1L shows the automation of a web matrix of porous frameworks of Stage 3 plantlets transferred to Stage 4 finishing media.

FIGS. 2A-B shows cross sections of a porous framework.

FIG. 2A shows a cross section of a porous framework having interstitial voids.

FIG. 2B shows a detailed, magnified cross section of voids.

FIGS. 3A-B shows details of interstitial void volumes.

FIG. 3A shows a detailed cross section of about 3 to about 40 ratio of large to small voids.

FIG. 3B shows a detailed cross section of about 5 to about 40 ratio of large to small voids.

FIGS. 4A-B shows in embodiments a porous framework having voids and nourishment solution distributed throughout.

FIG. 4A shows in embodiments a porous framework with a height.

FIG. 4B shows in embodiments a porous framework with a height.

FIGS. 5A-C shows the pocket of a porous framework in relation to an explant.

FIG. 5A shows a 3-dimensional view of a porous framework without an explant.

FIG. 5B shows an embodiment of a cross sectional view of a porous framework with an explant.

FIG. 5C shows an embodiment of a cross sectional view of a porous framework with an explant.

FIGS. 6A-B shows in embodiments details of surface contact between the explant and an improved support structure.

FIG. 6A shows a detailed cross section of about 15% surface area contact.

FIG. 6B shows a detailed cross section of about 38% surface area contact.

FIGS. 7A-B diagrams the relationship of the importance of the optimal nourishment solutions that influence capillary action and can increase yields.

FIG. 7A shows in embodiments how uniform capillary action may impact uniform distribution of plant growth hormones and nutrients.

FIG. 7B shows in embodiments how uniform distribution of plant growth hormones and nutrients may impact yield.

FIGS. 8A-B diagrams the impact of an improved support structure on increased yields which allows for automation.

FIG. 8A shows in embodiments how automation and increased yields due to improved support structure reduces labor and production costs which may increase profits.

FIG. 8B shows in embodiments how improved support structures result in increased yields which may allow for automation.

FIGS. 9A-B conceptually shows a distorted growth transport field and undistorted growth transport field.

FIG. 9A conceptually shows an embodiment of a distorted growth transport field.

FIG. 9B conceptually shows a embodiment of an undistorted growth transport field.

FIG. 10 conceptually shows the transplanting process from an explant container to a larger container.

FIGS. 11A-C shows embodiments of a transplant system.

FIG. 11A represents a transplant device, a dense population and a less dense population.

FIG. 11B represents a web matrix of growing explants.

FIG. 11C represents an embodiment of a transplant system.

FIG. 12 represents an embodiment of a transplant process.

### MODE(S) FOR CARRYING OUT THE INVENTION

As mentioned earlier, the present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application. Each of these aspects may at times be discussed separately or at times combined with other aspects in no particular order. It should be understood that all permutations and combinations are possible for any given system.

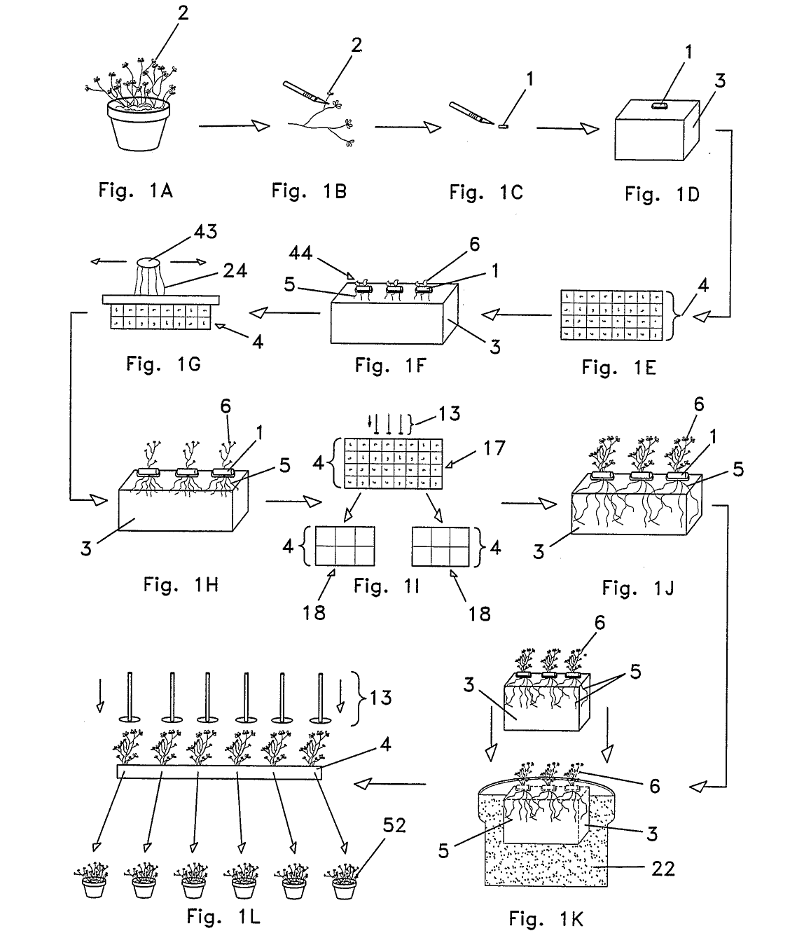

FIGS. 1A-L detail various embodiments of an overall tissue culturing process using a sustentacular tissue culturing devices, including a porous framework that could allow uniform distribution of plant growth hormones, nutrients and the like and allow for the use of automated processes and equipment to reduce labor costs.

As may be readily appreciated from FIGS. 1A through 1D, an explant (1) may be taken from a mother or stock plant (2) using traditional tissue culture techniques. Of course a propagule may be understood to be included in a tissue culturing process. An explant (1) may be placed on a tissue culturing growth media which may be an improved support structure, such as a porous framework (3) that can or can not be in a web matrix (4). This process may take place in a laboratory or other sterile environment to prevent contamination of the explant and porous framework by air bonie contaminates which may cause disease and reduce the potential yield of the explants harvested from the mother or stock plant.

A sustentacular tissue culturing device may support a tissue sample, such as an explant during the tissue culturing process. This processing may include, inter alia, supplying various kinds of nutrients and the like to an explant and growing the explant to a plantlet (52) and even a finished plant. By supplying solutions to an explant, it is understood that the nutrient solutions and the like solutions are in some way come into contact with an explant.

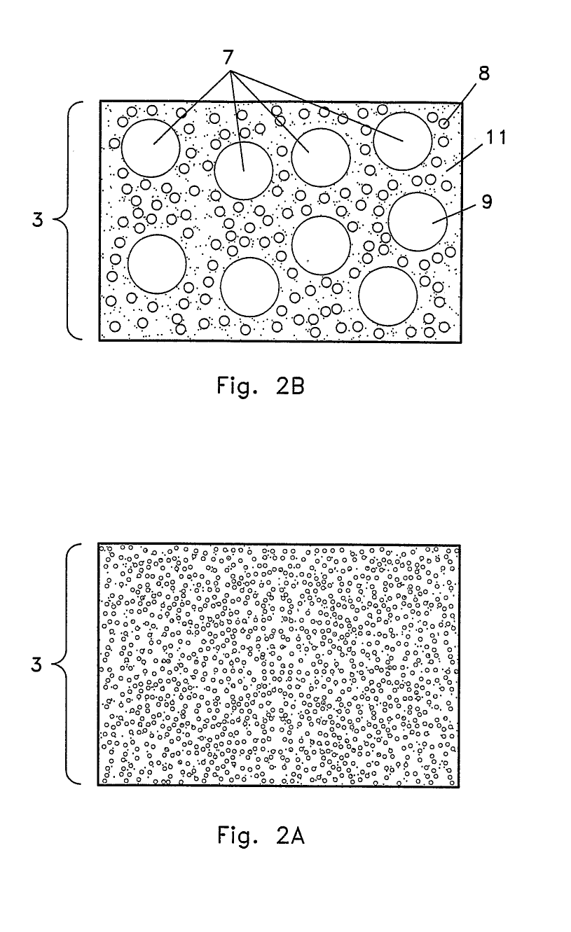

In embodiments, a porous framework (3) may be a skeletal structure that may be permeable by water, air, and the like. It should be understood that a porous framework does not include agar, a gelatin-like product, which may not be a skeletal structure. As can be seen in FIG. 2A, a porous framework (3) or even a multidirectional porous framework may be any type of porous structure. For example, but not being limited to, a porous framework may include a non-ceramic fiberous material, a non-gel structure, a foam, such as a wettable, open-celled polyurethane foam or even a phenol-formaldehyde resin, and the like structures. In embodiments, a porous framework may include, but is not meant to be limited to, peat moss, vermiculite, perlite, expanded foams, fiberous materials, either natural or manmade without unidirectional fibers such as cotton, stabilized organic and inorganic naturally occurring or manmade materials, eligaard or the like materials or even any combination of these materials.

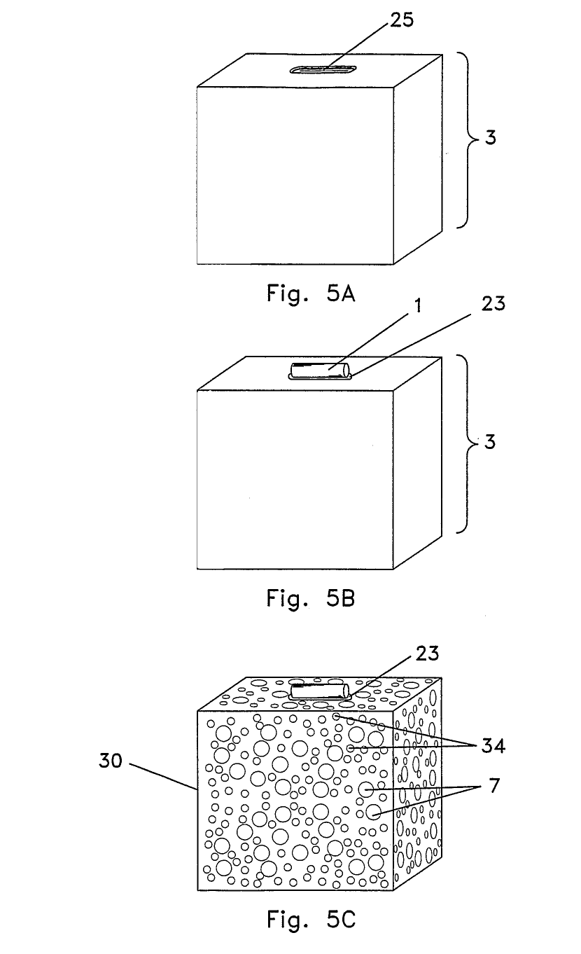

In other embodiments, the present invention may include an open surface multidirectional porous framework (30), as shown in FIG. 5C. Multidirectional may be a porous framework as defined herein that has multidirectional vectors (unlike having unidirectional vectors) within a framework, such as a sponge-like or web-like framework. An open surface may include having a surface, or even an upper surface that is not covered such as by a membrane, film, cover, or the like.

The present invention may include placing at least one explant on a surface of a porous framework. In embodiments, an explant may be placed in at least one pocket (which will be further described later). In yet other embodiments, the present invention may include at least one explant located on a surface of open surface multidirectional porous framework. The placement of an explant may be done manually or even automatically.

A porous framework (3) may be based on the specific species and/or variety requirements for proper development of root (5) and shoot (6) bud differentiation and development. A porous framework (3) may physically support a developing explant or plantlet by holding it in a proper orientation to light and perhaps even in an optimal orientation with a nourishment solution (24).

In embodiments, the present invention may include adding at least one nourishment solution (24) to a porous framework. The addition could include manually adding, automatically adding, and the like and could even be added by pouring, spraying, dripping, sprinkling, injecting and the like. A nourishment solution can include plant growth hormones, nutrients fertilizers, micro and macro nutrients for plant growth, vitamins, a source of carbohydrates, such as but not limited to sugar, and the like. A nourishment solution may be a gas, liquid, or solid and may even be liquid, solid or even gas solutions.

Of course throughout the growing process of the explant, in embodiments, more than one nourishment solution may be added to a porous framework. For example a first nourishment solution may be added to a porous framework and the explant may grow to at least an initial growth (e.g., buds of shoot and roots). A nourishment solution may be located near or even directly in contact with an explant so that an explant can sorb the solution. In embodiments, the first nourishment solution may be removed, and another nourishment solution may be added. The at least initially grown explant are then secondarily grown (e.g., further growth of shoots and roots).

A nourishment solution (24) may be supplied to an explant which may include having a nourishment solution close to an explant so that the explant may sorb the solution and grow. This may be achieved in different ways, such as but not limited to capillary action. A capillarity system may be a manifestation of surface tension by which a portion of a surface of a liquid coming in contact with a solid or the like may be elevated or depressed, depending on the adhesive or cohesive properties of the liquid. When a nourishment solutions has been supplied to an explant, the present invention provides, in embodiments, allowing an explant to sorb the nourishment solution. This includes the ability for an explant to intake the nourishment which can help the explant grow such as buds, shoots and roots. Of course, this may be accomplished by an explant sorbent element which includes the ability for the explant to sorb nourishment solutions.

As an explant may begin to mature it can grow on a porous framework. At least some of an explant, such as shoot buds, may grow above the framework and some of an explant, such as roots buds, may grow into the framework. Accordingly, the present invention may provide allowing an explant to grow that has been placed on a surface of the framework, yet also includes, as the explant begins to bud and shoot roots, growing within the framework, as shown in FIGS. 1A-L.

In order for the tissue culture cells to differentiate into root (5) and shoot (6) buds and then for the root (5) and shoot (6) buds to develop, it may require a correct distribution of plant growth hormones, nutrients and the like to be delivered to an explant (1). In embodiments, distribution of hormones and nutrients may be substantially uniform and may occur through capillary action. Substantially uniform may require the internal characteristics of a porous framework (3) to have certain ratios and percentages of size, proportion and relation. Further, in order for root (5) development to occur inside the porous framework (3), it may require certain ratios of air to moisture. Again, this may require that a porous framework's (3) internal characteristics have certain ratios and percentages of size, proportion and relation.

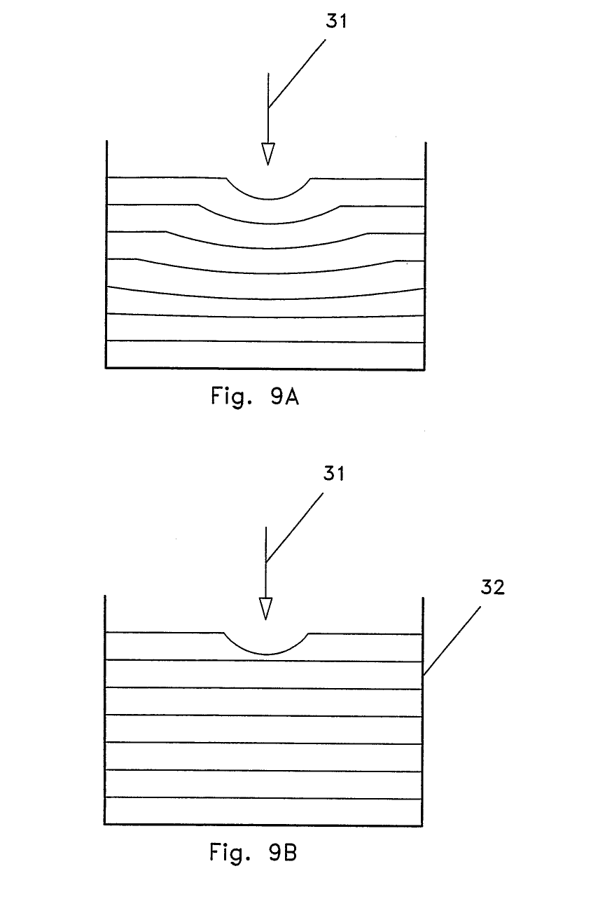

Referring to FIGS. 9A and 9B, conceptually, it can be seen how in embodiments an undistorted growth transport field (32) may be provided. When an explant is placed on a framework, or perhaps even when a pocket (25) may be created, the framework may be altered by such actions. For example, when a force (31) may be applied to certain materials, the applied force (which may include the placement of a tissue sample or the creation of a pocket) may distort the material, as shown in FIG. 9A. Of course FIGS. 9A and 9B are meant to only show conceptually how a growth transport field may be distorted. An actual framework when distorted may include other properties and distortions not shown. The distortion may effect the growth transport field of a framework including those areas at least close in proximity to where an explant may be located. A growth transport field may include air voids, a framework and the like. If a force is applied which distorts a field, air voids and a framework may also be distorted. Accordingly, with distorted air voids as well as a distorted framework, a nourishment solution may not adequately supply the nourishment solution to the explant. In the present invention, the material used in the porous framework, may include, in embodiments, an undistorted transport field (32) so that when a force is applied, the field (e.g., framework and air voids) may not change shape. In certain instances, if a pocket is made, it is done so without disturbance to the field. An undistorted growth transport field could allow maximum or even optimal conditions for supply of the nourishment solution to the explant.

In some embodiments, the present invention may include allowing a nourishment solution to move throughout an undistorted growth transport field. Capillary action may be utilized so that the solution can be distributed. In other embodiments, a porous structure may have an undistorted growth transport field adjacent to the explant. It may be important to provide an undistorted field near an explant, as well as near roots and the like and an explant begins to grow.

In other embodiments, the present invention may include a non-deformable structure (33). As discussed above, it may be desirable to have unaltered framework and air voids so that optimal nourishment and air may be provided to the explant as it grows. As such a non-deformable structure (33) may be any porous framework that cannot be substantially changed in shape or the like during the processing of a tissue culturing. Of course, some changes may occur to a non-deformable structure due to root buds and root growth. Accordingly, some yield may be appropriate during the tissue culturing process, yet it may be important to have an unaltered structure at least initially in the process.

As shown in FIG. 5C, the present invention may provide for extended interstitial voids (34) adjacent to an explant. This may include interstitial voids that are open, even fully open, and not disturbed in any way, e.g., due to an applied force or the like. An extended interstitial void (34) may be drawn out to its full length and may not be compressed or altered.

In embodiments, the invention may provide a porous framework that may contain consistent, uniform interspatial or even interstitial voids. The porous framework may be any tissue culturing material, such as but not limited to organic, inorganic, natural, manmade or the like materials that may be capable of providing consistent, uniform interstitial voids. The uniform interstitial voids may be necessary to allow even distribution and delivery of plant growth hormones, nutrients and the like to explants placed on them.

As seen in FIGS. 2A, 2B and 5C, the present invention may include defining a plurality of substantially uniform interstitial voids (7) within porous framework. Substantially uniform interstitial voids may be spaces or even air pockets between a framework. It should be understood that a void may be an open space in the absence of nutrient solutions and the like. Several uniform air pockets may be found within a porous framework or even within a multidirectional porous framework. The air pockets or even voids may vary in size somewhat. For example, in embodiments, substantially uniform interstitial voids may have a size difference of less than about 25%. Of course due to the variations and needs of different plants and species of plants, any size difference may be found in other embodiments and all are meant to be included in this disclosure.

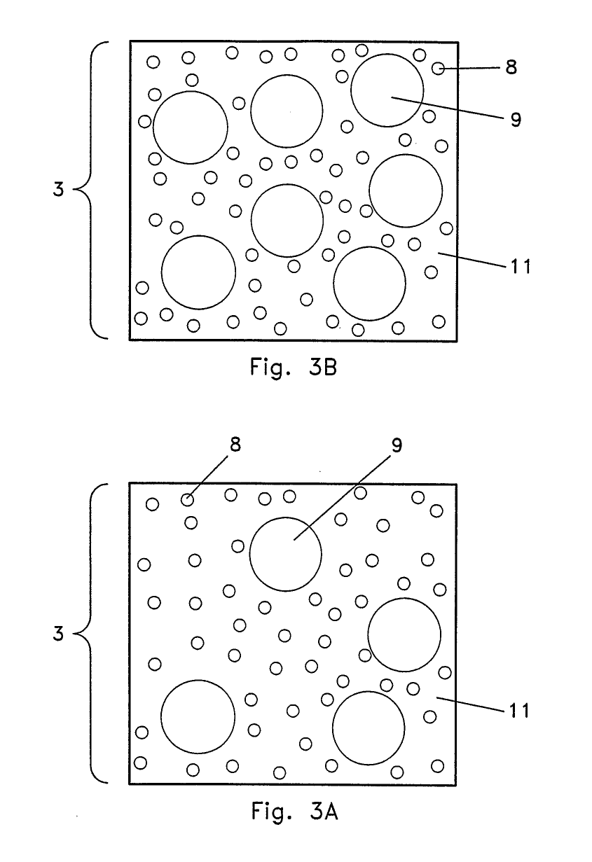

In some embodiments, the present invention may include defining at least some large and at least some small voids within a porous framework. This may include a ratio of large to small voids. Some examples of a ratio of large to small voids may include:
about 3 to about 40; and
about 5 to about 40.
   Of course any ratio may be used and is meant to be included in this disclosure. The ratio may be dependent upon the type of plant the may be used in the tissue culture. The ratio of large (9) interstitial voids to small (8) interstitial voids within the overall volume of interstitial voids may be important in order to maintain proper capillary action and perhaps to evenly distribute plant growth hormones and nutrients as shown in FIGS. 2B, 3A and 3B.

Yet, in other embodiments, the present invention may include a total void volume of a porous structure. Void volume could vary depending on specific species and/or variety requirements based on phenotypic and genotypic requirements of the specific species and/or variety. Void volume may be as low as about 10% or as high as about 60%. This could increase the proper development of root buds during Stage 1 and root formation during Stages 2 and 3. Improved root bud development and root formation could increase yields due to uniform development between explants within a group. This could allow a group of explants to move up to the next stage without grading which may be labor intensive and therefore expensive. Some examples of void volume may include:
about 10%;
about 20%;
about 30%;
about 40%;
about 50% and
about 60%.
   Of course, other void volumes may be used and are meant to be included in this disclosure. The void volumes may depend on individual species and/or variety requirements. With the correct volumes, maximum cell differentiation into root (5) and shoot (6) buds and consequently maximum development of the root (5) and shoot (6) buds may occur.

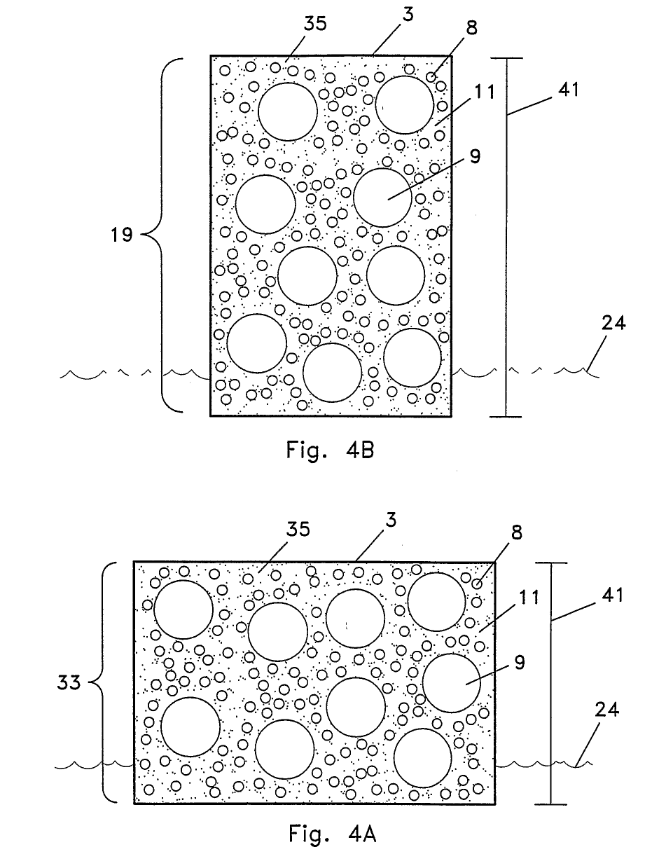

As shown in FIGS. 4A and 4B, another aspect of the invention may be that the height (41) of a porous framework may be dependent upon a void volume in the porous framework. In order to maintain proper concentrations of plant growth hormones, nutrients and the like at the top and throughout a porous framework, it may be necessary to have adequate capillary action of the liquid medium throughout a porous framework. Depending on the volume of the voids, the height (41) and even the width of a porous framework could vary. If a larger void volume is used, a shorter porous framework may need to be used because the capillary action with a large void volume could be reduced. An adequate height dependent upon void volume may increase uniformity of distribution and delivery of plant growth hormones, nutrients and the like to the explants and plantlets thereby possibly increasing yields of explants and plantlets. For example, if the void volume of a porous framework is high, the height of a framework may be shorter. On the contrary if the void volume is low, the height of a framework may be taller.

In embodiments, the present invention may include a porous framework having a size of about 15 mm in length by about 8 mm in width. Sizes of a porous framework may range from about 5 mm in length by about 2 mm in width to about 30 mm in length by 15 mm in width. Of course, a size of a porous framework may vary and may be dependent upon a void volume and even a size of interstitial voids, as previously discussed. In other embodiments, a sheet of porous frameworks may be used which may even enhance uniformity throughout the tissue culturing process. A sheet may be scored to break into individual pieces.

As shown in FIG. 2B, a porous framework may have a matrix of a continuous surface or even a framework (11) filled with interstitial voids (7). The interstitial voids (7) with the continuous surface area may make capillary action possible. In order for proper distribution of plant growth hormones, nutrients and the like to the explant (1) or plantlet, the correct proportion of continuous surface of a framework (11) with interstitial voids (7) may be necessary so that capillary action can occur. The proximity of the framework (11) to each other may cause a liquid's capillary action to rise vertically and horizontally through multidirectional porous framework. The size of the interstitial area between the continuous surfaces of the framework (11) may depend on the size and volume of the interstitial voids (7). In some embodiments, the interstitial voids (7) may not be equal in size or volume and may even vary depending on the type of improved support structure used. While the size of the interstitial voids (7) may be small (8) or perhaps even large (9), the amount of difference between small (8) and large (9) interstitial voids (7) may be not more than about 25%, as mentioned earlier. This may allow for the proper capillary action necessary to uniformly distribute the plant growth hormones, nutrients and the like to the developing explants (1) or plantlets.

When a nourishment solution is added to a porous framework, at least part of the voids may be filled with the nourishment solution. This may include allowing a nourishment solution to move throughout porous framework and at least some of substantially uniform interstitial voids, such as but not limited to capillary action. As previously discussed, in embodiments, to disperse a nourishment solution almost evenly throughout a porous framework, it may be desirable to have almost uniform interstitial voids to allow this even dispersion.

As mentioned before, more than one solution may need to be added to the explant and framework during the tissue culturing process. This may be done with a nourishment solution distributor (43). In embodiments, a first nourishment solution may be added to a porous framework and may be supplied or somehow brought near (including to) an explant.

With contact to a first solution, an explant may have at least an initial growth (44). This may include the beginning of shoot and root buds and may even include stage 1 of the tissue culture processing. After an amount of time, which may be determined by any number of factors including evaporation, plant growth, environment conditions, and the like, a second nourishment solution may be added. In embodiments, the present invention may include supplying a second nourishment solution to at least initially grown explants.

In embodiments, the present invention may include balancing retentive exchange capacities with removal exchange capacities of a nourishment solution in a porous framework. A retentive capacity may be the ability to retain or hold a nourishment solution within a porous framework. A removal capacity may be the ability to move or take away a nourishment solution. A balanced exchange between a solution held in a framework with the removal of the solution may be desirable. Some embodiments may include a nourishment solution exchange capacity and nourishment solution removal capacity balance element. For example, a first solution retained in a porous framework may be removed with a second nourishment solution. In embodiments, the present invention may allow for the rinsing of old solutions with new solutions as may be necessary to encourage cell differentiation and development of root (5) and shoot (6) buds.

In embodiments, the present invention may include affirmatively removing a first nourishment solution from a porous framework with a second nourishment solution. This may be achieved by an affirmative nourishment solution eliminator. Affirmatively removing or even the use of an affirmative nourishment solution eliminator may be the removal of all or maybe almost all of the first solution with a second nourishment solution. In yet other embodiments, the present invention may include substantially removing a first nourishment solution from a porous framework, or even a substantial nourishment solution remover element, which may includes removal of most if not all of a first nourishment solution.

In other embodiments, a nourishment solution may be added to a porous framework from above a porous framework. A system may include a nourishment solution distributor (43) located above an open surface multidirectional porous framework. This may allow quicker distribution of the solution and may even help with the affirmative removal of a first solution due to gravitational forces. Of course, other embodiments may provide for the addition of a solution other than above a porous framework. This may include but is not limited to injection, flooding, and the like.

Another embodiment may include providing a removal pressure of a nourishment solution greater than a retentive force of a nourishment solution. A removal pressure may include a pressure that is applied when adding a second nourishment solution. A retentive force may include the attraction, adhesive, or even cohesive and the like properties when a solution may be retained in the porous framework. It may be desirable to have a removal pressure greater than the retentive force to adequately remove most if not all of a first solution. This may be achieved in part due to gravity and the force of the addition of a new solution.

Nourishment solutions, including a first and second solutions, may be added to an explant on a porous structure automatically with perhaps an automatic nourishment solution distributor. This may include the technique, method, or system of operating or controlling a process by automatic systems, such as by electronic devices, which may reduce human intervention to a minimum. This may also include a mechanical device, operated electronically, that may function automatically, without continuous input from an operator.

In embodiments, a second nourishment solution could be a refresher solution containing the first solution components, or could be a different solution completely. This may be dependent upon the specific circumstances during the tissue culture process. A refresher solution may be needed to prevent a buildup of phenolic acid, which may be released by plant cells in response to the action of destroying cells during a cutting process. The phenolic acid may even become great enough to kill an explant. Refreshing could be based on the individual needs by species or variety. As but merely an example, a refresher solution may be added about 5 to 10 days after initially making an explant in Stage 1 or after cutting an explant during any of the subsequent stages, other times for addition is certainly possible.

In some situations and embodiments, the second solution may even be water. The present invention may provide a nourishment solution distributor which may include, but is not limited to a first nourishment solution distributor, a second nourishment solution distributor, a refresher nourishment solution distributor and the like distributors. The removal of old solutions and addition of new solutions may be repeated as often as desired and even as necessary.

A nourishment solution may be added to a porous framework by different ways of application. These may include, spraying, sprinkling, dripping, pouring, injecting and the like as previously stated. In other embodiments, the present invention may include a drain pan or a method for draining a nourishment solution from a framework. This may be used to remove an old nourishment solution from the framework or may even be used to prevent oversaturation of the framework, including any voids.

A porous framework may support an explant to ensure proper distribution of plant growth hormones, nutrients and the like. As discussed, an explant may be supplied with a nourishment solution in order to grow and mature. With proper distribution and delivery of plant growth hormones, a contact between a surface area of an explant (1) to a porous framework (3) may be critical for allowing the transfer of the plant growth hormones, nutrients and the like to an explant (1) allowing for cell differentiation and development of root (5) and shoot (6) buds.

In some embodiments, the present invention may include amply contacting at least part of an explant to a nourishment solution. Each porous framework could have a consistent or uniform pocket or indentation that can cradle the explants much like a pillow cradles a head while sleeping. One way of achieving this may be to provide a pocket (25) on a surface of a porous framework. A pocket (25) may be designed to provide optimal contact of an explant to hormones, nutrients and the like. The increased surface area of a porous framework that may be in contact with an explant may provide optimal conditions for successful propagation in a tissue culture environment. In embodiments the pocket may have a pocket size. Examples of a pocket size may include:
less than about 3.5 mm in length and about 2 mm in depth;
less than about 3 mm in length 1.5 mm in depth;
less than about 2.5 mm in length 1.5 mm in depth; and
less than about 2.0 mm in length 1.0 mm in depth.
   Of course any size is possible and is meant to be including with this disclosure.

In embodiments, the contact surface area of the explants to the contact surface area of the porous framework could be greater than about 15% and even less than about 38%. The contact surface area may increase the uniformity of development of the explants in each stage and may allow for transfer between stages without grading and could increase yields because immature explants may not be transferred before they have properly developed.

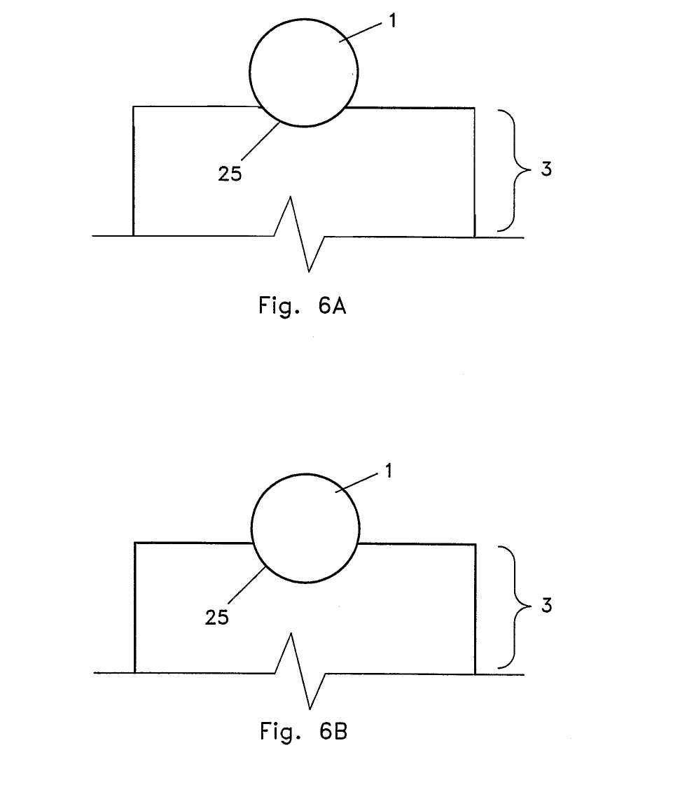

An explant may be placed in a pocket (25) and a nourishment solution may be added to the porous framework. The surface area contact between an explant and a pocket may provide for contact with the explant to the nourishment solution. As shown in FIGS. 5A, 5B and 5C, to have ample contact (23) between explant (1) and pocket (25) could include ample contact between explant and solution. As an example, ample contact (23) may include contacting an explant to a surface of pocket at a percentage contact value. A percentage contact value may include any percentage. Some of these may include:
greater than about 15%;
greater than about 20%;
greater than about 25%;
greater than about 30%; and
greater than about 35%.
   Of course, any percentage is intended to be included in this disclosure. Examples of the various contacts can seen in FIGS. 6A and 6B.

In embodiments, the present invention may include substantially uniformly distributing nourishment solution (35) throughout a porous framework, as may be seen in FIGS. 4A and 4B. By substantially uniformly distributing it is meant to include consistently or even mostly identically spreading a nourishment solution in a framework. In embodiments, each part of a framework may have almost the same if not the same amount of nourishment solution which is evenly distributed throughout a framework. Of course a perfectly even distribution may not occur, so a substantially uniform distribution may occur which may include almost perfectly or even almost equally distributing nourishment solution throughout a porous framework. Embodiments may include devices such as an open surface multidirectional porous framework which is capable of substantial uniform distribution or even an almost equal distribution of a nourishment solution.

In embodiments, the present invention may include providing and maintaining sufficient exposure of air to an explant. Of course as the explant grows it may need to be in contact with air. Initially, part of the explant may be situated in air and part may be situated on or even in a porous framework. The framework may be partly saturated with a nourishment solution or may be fully saturated with a nourishment solution. As the explant grows, the roots and the growth that takes place within the framework could be exposed to a solution. In order to prevent the growing explant from drowning, at least some air may need to be in the framework. A balance between air and nourishment solution may be desirable so that explant and its growth may have sufficient exposure to air and nourishment.

Interstitial voids (7), as previously discussed, may provide air to the developing roots (5). In embodiments, the present invention may provide balancing air to nourishment solution in an air volume to liquid mass ratio. The amount of air and moisture may be dependent on the individual species and/or variety for optimal development.

The amount of liquid retained in a framework may be a function of the size and volume of the voids. Many small voids could hold more liquid than a few large voids. The surface tension of a liquid may also determine how much saturation of the voids could occur.

The present invention may provide, in embodiments, optimally balancing air to nourishment solution within a porous framework. In general, ratio of 50% air to 50% liquid may be optimal for successful root formation and development. This could of course vary by species (e.g., a cactus could require less liquid, whereas a water lily could need more liquid than a cactus). An example of the range of ratios of air to nourishment solution may include;
about 20% air to about 80% nourishment solution;
about 30% air to about 70% nourishment solution;
about 40% air to about 60% nourishment solution;
about 50% air to about 50% nourishment solution;
about 60% air to about 40% nourishment solution;
about 70% air to about 30% nourishment solution; and
about 80% air to about 20% nourishment solution.
   Other ratios are possible and are meant to be included in this disclosure.

The amount of liquid or nourishment solution may be based upon the requirements of a species. The quantity of nourishment solution may be based, in embodiments, on the void size and volume of a porous framework. Less liquid may be needed if there are few, small voids. More liquid may be necessary for many large voids. In yet other embodiments, the air in the framework may be reduced substantially, saturating a framework to reduce the air void volume which may reduce and even suppress root formation and development.

In embodiments, the present invention may include preventing vitrification of an explant where an explant may have a translucent water soaked succulent appearance which may leads to mortality. It may be desirable to provide and maintain sufficient exposure of an explant to light as the explant grows. This may include providing a light source (such as but not limited to the sun, a sun lamp, and the like) near the explant.

Automation could allow for the easy transfer of multiple explants or plantlets between stages that may even decreased production costs. In embodiments, uniformity may be critical for automated transfer of multiple explants or plantlets to prevent the transfer of immature or overly mature explants in the same transfer.

Automation could also allow for a more efficient use of expensive laboratory or sterile space during at least the first stages of the tissue culture process. By utilizing a more dense (17) population spacing initially, less overall laboratory or sterile area could be required. Then, as an explant or plantlet matures and subsequently becomes larger, the explants or plantlets may be moved to a less dense (18) population spacing.

In embodiments, a porous framework may allow physical movement of at least part of a porous framework with an explant, growing explant or even a plantlet (52). Transfer of explants such as from one stage to another may include processes and procedures that employ machines that may automatically move at least part of a porous framework and an explant located on a porous framework to a new location. This new location may allow for new environmental properties such as light, humidity, temperature and the like. Equipment may also move explants from a high density of explants or explants per cm² to a lower density of explants or explants per cm² to allow for the natural growth and increased size of the explants as the root and shoot buds develop into plantlets (52). The equipment may be designed to handle multiple explants or plantlets at a time which may further increase the efficiency of the transfer process. This could greatly improve the efficiency of not only the labor to transfer between stages, but also may reduce the required space in a laboratory or sterile environment that may be highly expensive due to the nature of being a laboratory, sterile environment and even a specialized area. Therefore, more explants may be brought to maturity in Stage 4, increasing yield, possibly because of increased uniformity throughout the tissue culture process.

In some tissue culturing systems, it may be desirable to transfer a growing explant in a first environment (62) to a new environment. One of the reasons for doing this may be to move a dense population of explants into a less dense population as the explants grow and need more space. This may be sensible in order to save space earlier in the tissue culturing processing among other reasons. After an explant has been placed in a first environment (62), it begins to grow. A transplant growth criterion may be determined at which time, when the explant meets the criteria, it could be moved or transplanted to a new environment. A transplant growth criterion may be specific to the type of plant species and thus, there may be different growth criterion for each species and even many criterion to be used with one species. A transplant growth criterion may include, for example, when the explant has grown to a certain size. The explants may even be transplanted more than once during the tissue culturing process and may even be transplanted when they have matured into a plantlet such as during stage 4. As such, the present invention may include determining at least one transplant growth criterion appropriate to a given plant species.

In embodiments, a first environment (62) may include a tissue culture growth media and a plurality of explants. As an example, a tissue culture growth medium may include a porous framework or even an open surface multidirectional porous framework. The explants may be nurtured to at least an initial growth (44). This may include initial beginning of shoot and root buds to maturing shoots and roots and even mature shoots and roots. In embodiments, the present invention may include placing a plurality of explants on a surface of a porous framework. Further, in embodiments, the addition of at least one nourishment solution to a tissue culture growth media, or in fact to a porous framework and explant may be included. These systems may include placing a tissue culture growth media and a plurality of explants in a dense population which may include spacing the explants closely together.

When a substantial portion of the explants has grown to meet a transplant growth criterion, the transplant growth criterion may be established. This may include some or even most, or even yet all of the explant meeting the criteria. In other embodiments, an affirmative establishment of a transplant growth criterion may be included so that a substantial portion of a plurality of initially grown explants while situated in first environment may meet a transplant growth criterion. An enhanced yield may even be statistically increased by merely affirmatively establishing the criterion and then accomplishing the transplant event at a time when that criterion is substantially established.

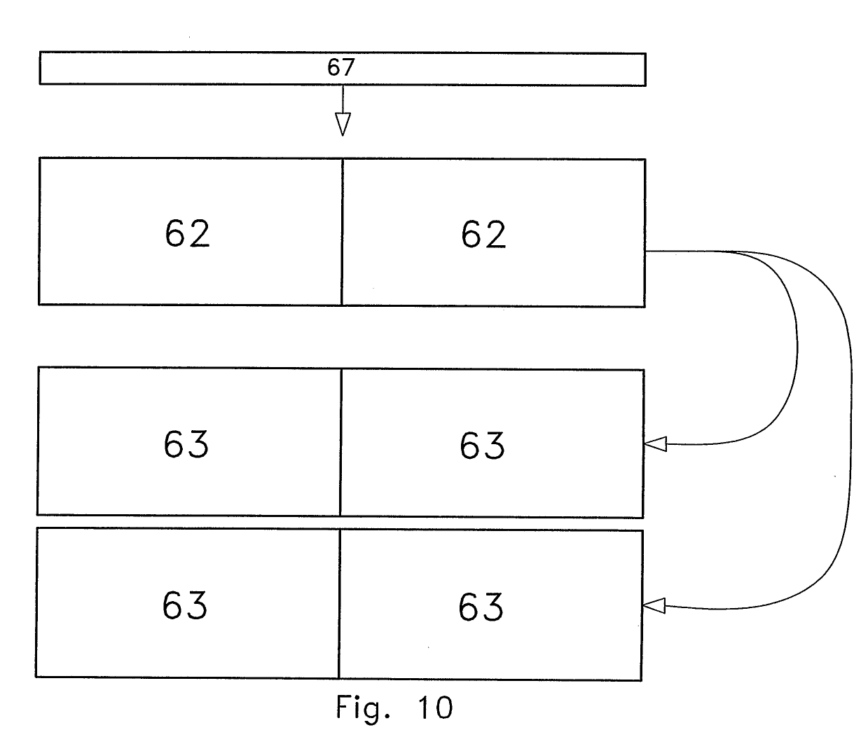

In embodiments, the present invention may include extruding the initially grown explants and at least some of the tissue culture media from a first environment at a time when transplant growth criterion may be substantially established. The initially grown explants and at least some of the tissue culture media may be inserted from the first environment into a second environment (63) immediately after the extrusion. The explants placed in a second environment (63) may be spaced in a less dense population as the first environment, as shown conceptually in FIG. 10. In the second environment (63), the initially grown explants can secondarily grow. A nourishment solution may be added to a second environment and this process may be repeated as many times as desirable.

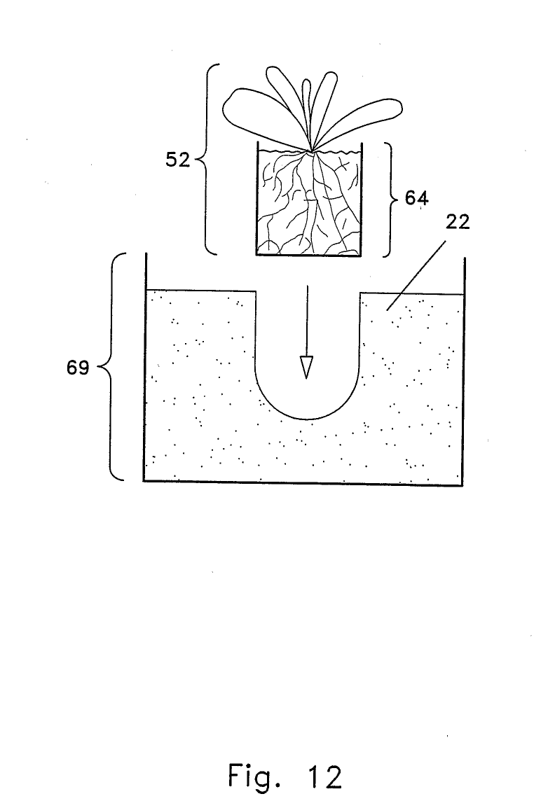

As an explant develops and grows roots (5) into a porous framework (3), the roots may anchor the growing explant (1) or plantlet to the porous framework (3) which may contribute to an effective transplant. The present invention, in other embodiments, may include supplying a synthetic retentive capability (64), as may be shown in FIG. 12. A synthetic retentive capability (64) may include an artificial, non-natural or even manufactured structure or material that has an ability to retain its shape and structure. The present invention provides for maintaining a synthetic retentive capability during an extrusion and insertion processes, as mentioned above. This may be notable so that the explant may be transplanted without damage to it, with less difficulty, and the like.

It may be sensible to properly balance a synthetic retentive capability (64) of a tissue culture media or even a porous structure with a plant yield ability (65). A balance allows a porous structure to move when roots grow from an explant, yet allows a porous structure to keep its shape when it is transferred into a new environment.

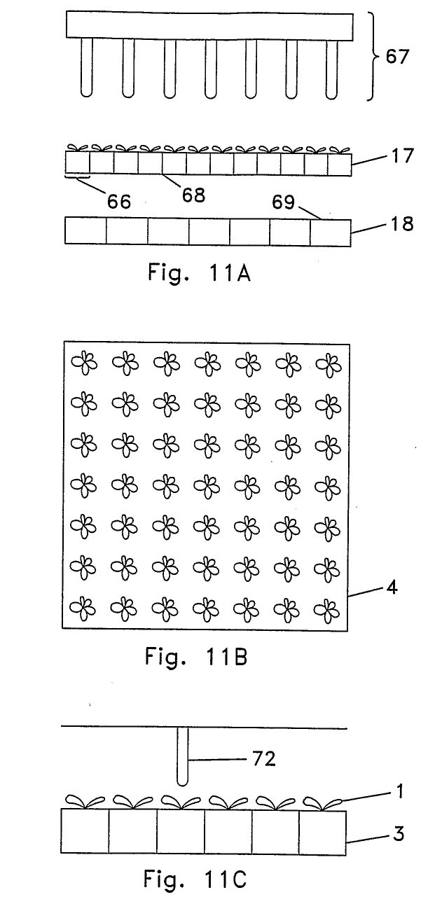

In some embodiments, the tissue culture growth media and plurality of explants may be placed in a matrix of transplant containers (66) or even a first matrix of explant transplant containers as shown in FIG. 11A.

In one modality, it is possible that in both extruding and inserting an explant, this action can occur continually, that is, as part of a single step which both pushes an explant out and as part of the same uninterrupted motion pushes it into a new container. Thus, the system may be arranged as a continuate insert system. This may occur immediately after extruding the explant. Multiples of the extrusion and insertion processes for a plurality of explants can occur at once and even simultaneously for even more efficiency.

Especially appropriate to the invention is using a system which provides for simultaneous transplantation of a plurality of explants or even plantlets at once. This may include simultaneously extruding (such as through a simultaneous extrusion system) and/or simultaneously inserting (such as through a simultaneous insertion system), each as represented in embodiments in FIGS. 10 and 11A. All this may be accomplished through an automatic transplant system, of course.

In other systems, the process of transferring an explant as described in embodiments above, may be automated. This may include automatically placing a plurality of explants in a first environment, automatically extruding and inserting the explants and tissue culture media, and the like.

Since explants may be planted perhaps in a first matrix, it may be deemed appropriate to transfer the explants to a larger container, often using a punch-transplant device (67). In a punch down system, this is usually accomplished by using a plant punch element (72) to act upon an explant (1) and at least part of a porous framework (3), as shown in FIGS. 11A and 11C. The plant punch element (72) thus causes a substantial portion—if not all—of the explant (1) and at least part of a porous framework (3) to be extruded from a transplant container (66) through a yieldable exit element (68) or the bottom of each container. By permitting the plant punch element (72) to have movement within or even through a web matrix (4), the extruded explant (1) and at least part of a porous framework (3) may be placed in post transplant containers (69). This can occur, in embodiments, because most of the explant and porous framework are cohesive and thus present an individual transplant cohesive plant mass. Of course the matrix may also be arranged in a rectilinear matrix of orderly rows and columns.

Another objective of the invention may include a plurality of explant transplant containers (66) within which an explant growth may be impacted by a punch-transplant device (67) as shown in FIGS. 11A and 11C. Explant transplant containers (66) may contain a tissue culture growth medium as well as a plurality of explants. The explants may be responsive to the tissue culture growth medium. The explant transplant containers may have a yieldable exit element (68) that allows the tissue culture growth medium and explant to be pushed through the container. An explant transplant container may contain a nourishment solution. The explant transplant containers may include a dense population of plurality of explants. After transplanting, the explants may be moved into post transplant containers (69) that may be in a less dense population than the explant transplant containers may have been.

An explant may remain on a porous structure and grow until it becomes an plantlet. The present invention, in embodiments, may include placing a plantlet and at least some of a porous framework in a new medium (22). A new medium may include soil, peat moss, peat, bark, inorganic substances, organic substances, gravel, sand, natural substances, man-made substances, clay, liquid, finishing media, prefinishing media combinations of these, other finishing or prefinishing media as may be well understood by those familiar in the art and the like.

Surprisingly, when a porous framework in transferred into a new medium, the present invention may include providing a porous framework that can disperse and even dissolve into the new medium over time. It may be desirable to provide a porous framework that can disintegrate when it is transferred into a new medium.

Optimum capillary action could produce highly uniform explants and plantlets which may facilitate the use of automation (13) for the transfer process. Automatic equipment may require consistent uniformity of cell differentiation and development (14) in order to maintain efficiency. Uniformity could also increase yield (15) of finished plants from the initial explants taken. The higher the yield (15) from beginning to end, the greater the efficiency and the lower the production costs (16) per finished plant may occur. Lower yields may indicate ununiform or non-uniformity which may result in grading by hand based on maturity or characteristics necessary before transfer to the next Stage. Manual grading may increase labor costs and may increase overall time which can dramatically increase production costs.

In some embodiments a porous framework may be an only porous framework or even an only open surface multidirectional porous framework. This may include that nothing has been added to is present in a framework, other than the framework and voids. Other solution retention elements or the like such as gel are excluded from an only porous framework. This of course, does not exclude nutrients and solutions that may be added during the tissue culturing processes in order to facilitate the explants to grow.

Other objectives of another embodiment of the invention may include placing a plurality of explants on a surface of a plurality of porous frameworks arranged in a web matrix (4) as shown in FIG. 11B. Other objectives of yet another embodiment of the invention may include uniformly growing a plurality of explants. This may be desirable to increase yield of the total number of explants that mature into plantlets and may even provide maturing the explants at a substantially similar rate. In embodiments, the present invention may include providing substantially similar conditions for each of plurality of explants such as but not limited to providing substantially similar explant specimens or even providing substantially similar contact of explants to at least one nourishment solution or even to a pocket or yet even utilizing a controlled environment.

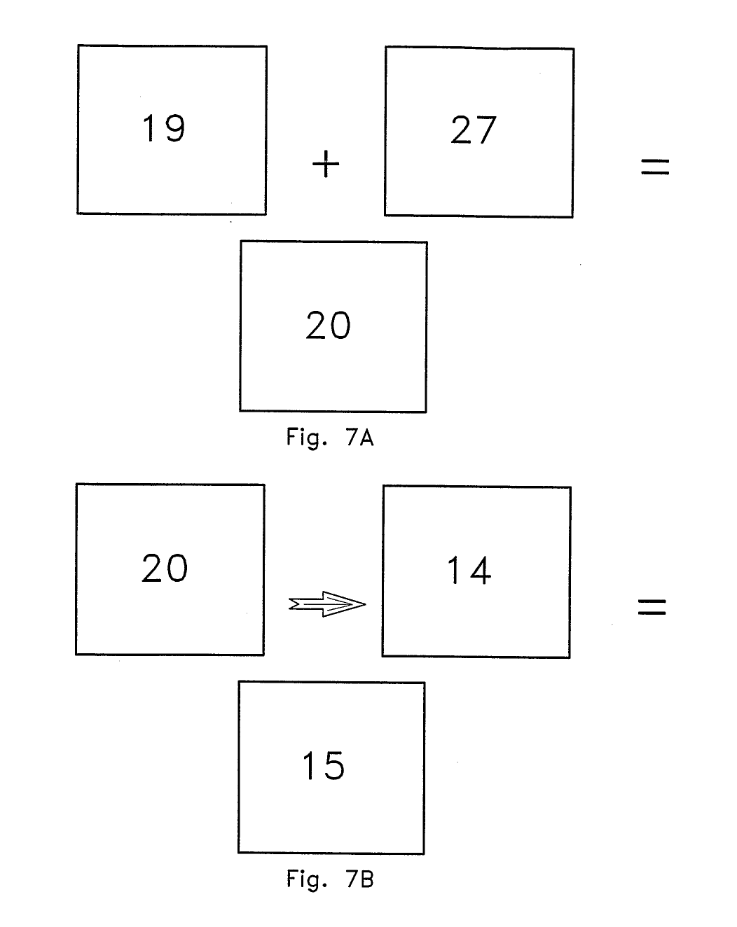

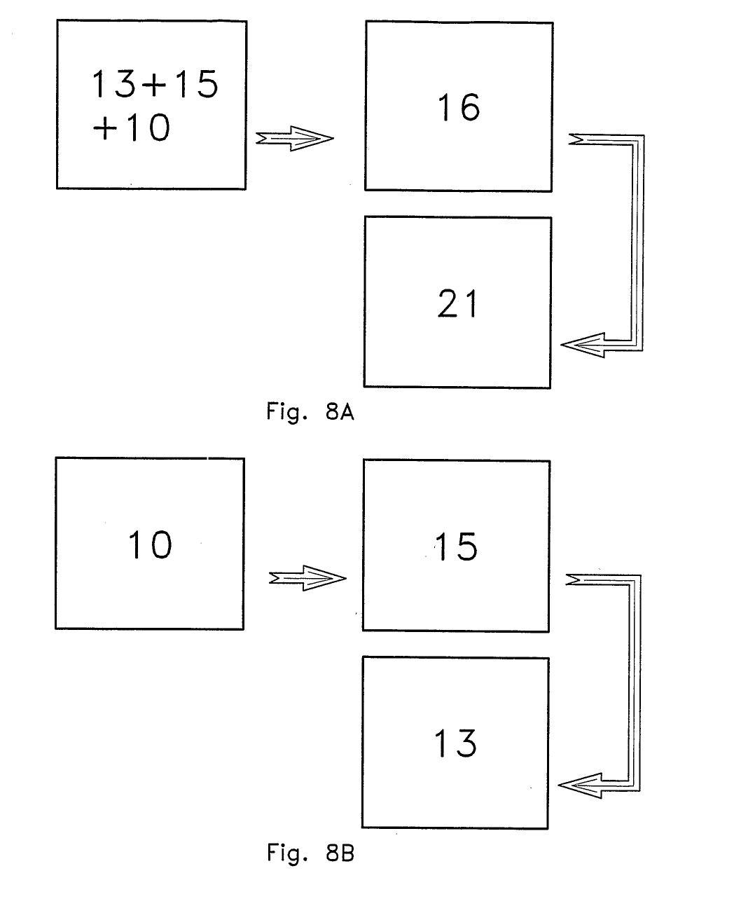

Referring to FIGS. 7A, 7B, 8A and 8B, the invention's attributes of an improved support structure or porous framework with uniform capillary action (19) in addition to optimal concentrations of plant growth hormones and nutrients (27) may result in uniform distribution of plant growth hormones, nutrients (20) and the like. Uniform distribution of hormones, nutrients (20) and the like may result in consistent, uniformity of cell differentiation and development (14) of explants and plantlets. The consistent, uniform cell differentiation and development (14) of explants and plantlets may increase yields (15). Automation (13) and increased yields (15) or even achieving increased population yields due to improved support structures (10), such as porous frameworks and the like as described herein in various embodiments, may reduce labor and lower production costs (16) which may result in an overall increase in profits (21). An improved support structure (10) therefore may result in increased yields (15) and may allow for automation (13) processes.

As can be easily understood from the foregoing, the basic concepts of the present invention may be embodied in a variety of ways. It involves both tissue culture techniques as well as devices to accomplish the appropriate tissue culture. In this application, the tissue culture techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims in this or any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used, the term “element” is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “supply” should be understood to encompass disclosure of the act of “supplying”—whether explicitly discussed or not—and, conversely, were there effectively disclosure of the act of “supplying”, such a disclosure should be understood to encompass disclosure of a “supply” and even a “means for supplying.” Such changes and alternative terms are to be understood to be explicitly included in the description.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the chart below or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

I. U.S. PATENT DOCUMENTS

| DOCUMENT | | | | | FILING |
|---|---|---|---|---|---|
| NO. & KIND | PUB'N DATE | PATENTEE OR | | | DATE |
| CODE | mm-dd-yyyy | APPLICANT NAME | CLASS | SUBCLASS | mm-dd-yyyy |
| 3,447,261 | 06/03/1969 | Hundt | 47 | 34.13 | 10/05/1966 |
| 3,755,964 | 09/04/1973 | Rack | 47 | 37 | 01/07/1972 |
| 3,799,078 | 03/26/1974 | Blackmore et al. | 111 | 2 | 04/19/1972 |
| 3,820,480 | 06/28/1974 | Blackmore et al. | 111 | 2 | 09/17/1973 |
| 4,377,639 | 03/22/1983 | Lee | 435 | 285 | 01/18/1982 |
| 4,531,324 | 07/30/1985 | Yang et al. | 47 | 81 | 10/07/1983 |
| 4,586,288 | 05/06/1986 | Walton | 47 | 73 | 07/18/1983 |
| 4,910,146 | 03/20/1990 | Tur-Kaspa et al. | 435 | 284 | 07/18/1988 |
| 4,947,579 | 08/14/1990 | Harrison et al. | 47 | 1.01 | 10/19/1988 |
| 4,947,582 | 08/14/1990 | Visser, Anthony | 47 | 101 | 07/13/1989 |
| 4,970,824 | 11/20/1990 | Visser | 47 | 86 | 01/09/1989 |
| 4,998,945 | 03/12/1991 | Holt et al. | 47 | 1.01 | 12/07/1989 |
| 5,048,434 | 09/17/1991 | Forster et al. | 111 | 105 | 04/23/1990 |
| 5,088,231 | 02/18/1992 | Kertz | 47 | 1.01 | 08/24/1990 |
| 5,141,866 | 08/25/1992 | Levin | 435 | 240.45 | 06/22/1988 |
| 5,225,345 | 07/06/1993 | Suzuki et al. | 435 | 284 | 07/19/1991 |
| 5,247,761 | 09/28/1993 | Miles et al. | 47 | 1.01 | 01/03/1991 |
| 5,257,889 | 11/02/1993 | Suzuki et al. | 414 | 417 | 11/27/1991 |
| 5,295,325 | 03/22/1994 | Honda et al. | 47 | 1.01 | 01/14/1991 |
| 5,320,649 | 06/14/1994 | Holland | 47 | 1.01 | 08/18/1992 |
| 5,365,693 | 11/22/1994 | Van Wingerden et al. | 47 | 1.01 | 09/10/1992 |
| 5,370,713 | 12/06/1994 | Hanseler | 47 | 1.01 | 09/05/1991 |
| 5,425,202 | 06/20/1995 | Mekler | 47 | 58 | 07/27/1993 |
| 5,488,802 | 02/06/1996 | Williames | 47 | 101 | 03/07/1990 |
| 5,536,281 | 07/16/1996 | Lambert | 47 | 1.01 | 12/13/1994 |
| 5,548,924 | 08/27/1996 | Mekler | 47 | 69 | 06/02/1995 |
| 5,842,306 | 12/01/1998 | Onosaka et al. | 47 | 1.01 | 11/16/1995 |
| 5,860,372 | 01/19/1999 | Bouldin et al. | 111 | 105 | 09/23/1996 |
| 5,867,937 | 02/09/1999 | Templeton | 47 | 59 | 06/24/1997 |
| 5,911,631 | 06/15/1999 | Bouldin et al. | 47 | 1.01 | 01/30/1997 |
| 6,044,778 | 04/04/2000 | Shokaku et al. | 111 | 105 | 01/14/1998 |
| 6,079,153 | 06/27/2000 | Templeton | 47 | 59 | 07/28/1998 |
| 6,212,821 | 04/10/2001 | Adam et al. | 47 | 1.01 | 05/10/1999 |
| 6,381,901 | 05/07/2002 | Friedman | 47 | 79 | 11/08/1999 |
| 6,391,638 | 05/21/2002 | Shaaltiel | 435 | 383 | 02/08/1999 |
| 6,479,433 | 11/12/2002 | Hann et al. | 504 | 141 | 10/02/2000 |
| 6,576,458 | 06/10/2003 | Sarem et al. | 435 | 286.5 | 09/20/2000 |

II. FOREIGN PATENT DOCUMENTS

| Foreign Patent Document | | |
|---|---|---|
| Country Code, Number, | PUB'N DATE | PATENTEE OR |
| Kind Code | mm-dd-yyyy | APPLICANT NAME |
| DE 2843905 A1 | 04/24/1980 | Hoelter |
| DE 3207623 A1 | 09/29/1983 | |
| EP 0117766 A1 | 09/05/1984 | Challet |
| EP 0692929 B1 | 02/04/1998 | Tanny |
| WO 87/00394 A1 | 01/29/1987 | Nippon Steel |
| | | Chemical Co. |
| WO 96/33845 A1 | 10/31/1996 | Alper |
| WO 02/058455 A1 | 08/01/2002 | Tagawa |
| WO 02/100159 A2 | 12/19/2002 | Tagawa |

Thus, the applicant(s) should be understood to have support to claim and make a statement of invention to at least: i) each of the tissue culture systems as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, and xii) each potentially dependent claim or concept as a dependency on each and every one of the independent claims or concepts presented.

With regard to claims whether now or later presented for examination, it should be understood that for practical reasons and so as to avoid great expansion of the examination burden, the applicant may at any time present only initial claims or perhaps only initial claims with only initial dependencies. Support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1-24. (canceled)

25. A method of tissue culturing processing comprising the steps of:
placing at least one explant in at least one pocket on a surface of a porous framework;
adding a first nourishment solution to said porous framework;
supplying said first nourishment solution to said explant;
growing at least an initial growth of said explant on said porous framework;
adding a second nourishment solution to said porous framework;
balancing retentive exchange capacities with removal exchange capacities of said first nourishment solution in said porous framework;
affirmatively removing said first nourishment solution from said porous framework with said second nourishment solution; and
secondarily growing said at least initially grown explants.

26-32. (canceled)

33. A method of tissue culturing processing comprising the steps of:
determining at least one transplant growth criterion appropriate to a given plant species;
placing a tissue culture growth media and a plurality of explants in a first environment;
nurturing at least an initial growth of said explants in said first environment;
establishing said at least one transplant growth criterion for a substantial portion of said plurality of initially grown explants while situated in said first environment;
extruding said initially grown explants and at least some of said tissue culture media from said first environment at a time when said transplant growth criterion is substantially established;
inserting said initially grown explants and at least some of said tissue culture media from said first environment in a second environment immediately after extruding said initially grown explants and at least some of said tissue culture media from said first environment; and
secondarily growing said initially grown explants.

34. A method of tissue culturing processing according to claim 33 and further comprising the steps of
supplying a synthetic retentive capability; and
maintaining said synthetic retentive capability during said step of extruding said initially grown explants and at least some of said tissue culture media from said first environment at a time when said transplant growth criterion is substantially established and said step of inserting said initially grown explants and at least some of said tissue culture media from said first environment in a second environment immediately after extruding said initially grown explants and at least some of said tissue culture media from said first environment.

35. A method of tissue culturing processing according to claim 34 and further comprising the step of properly balancing said synthetic retentive capability with a plant yield ability.

36. A method of tissue culturing processing according to claim 33 wherein said step of placing a tissue culture growth media and a plurality of explants in a first environment comprises the step of placing said tissue culture growth media and a plurality of explants in a first matrix of transplant containers.

37. A method of tissue culturing processing according to claim 33 wherein said step of establishing said at least one transplant growth criterion for a substantial portion of said plurality of initially grown explants while situated in said first environment comprises the step of affirmatively establishing said at least one transplant growth criterion for a substantial portion of said plurality of initially grown explants while situated in said first environment.

38. A method of tissue culturing processing according to claim 33 wherein said steps of extruding said initially grown explants and at least some of said tissue culture media from said first environment at a time when said transplant growth criterion is substantially established and inserting said initially grown explants and at least some of said tissue culture media from said first environment in a second environment immediately after extruding said initially grown explants and at least some of said tissue culture media from said first environment comprises the step of simultaneously extruding said initially grown explants and at least some of said tissue culture media from said first environment at a time when said transplant growth criterion is substantially established and simultaneously inserting said initially grown explants and at least some of said tissue culture media from said first environment in a second environment immediately after extruding said initially grown explants and at least some of said tissue culture media from said first environment.

39. A method of tissue culturing processing according to claim 33 wherein said step of inserting said initially grown explants and at least some of said tissue culture media from said first environment in a second environment immediately after extruding said initially grown explants and at least some of said tissue culture media from said first environment comprises the step of continually inserting said initially grown explants and at least some of said tissue culture media from said first environment in a second environment immediately after extruding said initially grown explants and at least some of said tissue culture media from said first environment.

40. A method of tissue culturing processing according to claim 33 wherein said step of nurturing at least an initial growth of said explants in said first environment comprises the step of adding at least one nourishment solution to said tissue culture growth media and said explants.

41. A method of tissue culturing processing according to claim 33 wherein said step of placing a tissue culture growth media and a plurality of explants in a first environment comprises the step of placing said tissue culture growth media and said plurality of explants in dense population.

42. A method of tissue culturing processing according to claim 33 wherein said step of inserting said initially grown explants and at least some of said tissue culture media from said first environment in a second environment immediately after extruding said initially grown explants and at least some of said tissue culture media from said first environment comprises the step of inserting said initially grown explants and at least some of said tissue culture media from said first environment in a less dense population than said first environment immediately after extruding said initially grown explants and at least some of said tissue culture media from said first environment.

43. A method of tissue culturing processing according to claim 33 and further comprising the steps of
growing said explant into a plantlet; and
placing said plantlet into a new medium selected from the group consisting of soil, peat moss, peat, bark, inorganic substances, organic substances, gravel, sand, natural substances, man-made substances, clay, liquid, finishing media, and prefinishing media.

44-59. (canceled)

60. A method of tissue culturing processing according to claim 33 wherein said step of placing a tissue culture growth media and a plurality of explants in a first environment comprises the step of placing said plurality of explant on a surface of a porous framework and wherein said step of nurturing at least an initial growth of said explants in said first environment comprises the step of adding at least one nourishment solution to said porous framework.

61. A method of tissue culturing processing according to claim 60 and further comprising the step of substantially uniformly distributing said at least one nourishment solution throughout said porous framework.

62. A method of tissue culturing processing according to claim 61 wherein said step of substantially uniformly distributing said at least one nourishment solution throughout said porous framework comprises the step of almost equally distributing said at least one nourishment solution throughout said porous framework.

63. (canceled)

64. A method of tissue culturing processing according to claim 60 and further comprising the step of amply contacting at least part of said explant in said pocket to said at least one nourishment solution.

65. A method of tissue culturing processing according to claim 64 wherein said step of amply contacting at least part of said explant in said pocket to said at least one nourishment solution comprises the step of contacting said at least one explant to a surface of said pocket at a percentage contact value, said percentage contact value selected from the group consisting of:
greater than about 25%;
greater than about 30%; and
greater than about 35%.

66. (canceled)

67. A method of tissue culturing processing according to claim 60 wherein said step of adding at least one nourishment solution comprises the step of adding a first nourishment solution to said porous framework.

68. A method of tissue culturing processing according to claim 67 and further comprising the steps of:
adding a second nourishment solution to said porous framework;
balancing retentive exchange capacities with removal exchange capacities of said first nourishment solution in said porous framework; and
affirmatively removing said first nourishment solution from said porous framework with said second nourishment solution.

69. A method of tissue culturing processing according to claim 68 wherein said step of balancing retentive exchange capacities with removal exchange capacities of said first nourishment solution in said porous framework comprises the step of providing a removal pressure of said first nourishment solution greater than a retentive force of first nourishment solution to said porous framework.

70. A method of tissue culturing processing according to claim 68 wherein said step of affirmatively removing said first nourishment solution from said porous framework with said second nourishment solution comprises the step of substantially removing said first nourishment solution from said porous framework.

71. (canceled)

72. A method of tissue culturing processing according to claim 68 wherein said step of adding a second nourishment solution to said porous framework comprises the step of adding a refresher solution of said first nourishment solution to said porous framework.

73. A method of tissue culturing processing according to claim 60 and further comprising the step of defining a plurality of substantially uniform interstitial voids within said porous framework.

74. A method of tissue culturing processing according to claim 73 wherein said step of defining a plurality of substantially uniform interstitial voids within said porous framework comprises the step of defining a plurality of substantially uniform interstitial voids having a size difference of less than about 25%.

75. A method of tissue culturing processing according to claim 73 wherein said step of defining a plurality of substantially uniform interstitial voids within said porous framework comprises the step of defining at least some large and at least some small voids.

76. A method of tissue culturing processing according to claim 75 wherein said step defining large and small voids comprises the step of providing a ratio of said large to small voids selected from the group consisting of:
about 3 to about 40; and
about 5 to about 40.

77. (canceled)

78. A method of tissue culturing processing according to claim 60 and further comprising the step of providing an undistorted growth transport field of said porous framework.

79. (canceled)

80. A method of tissue culturing processing according to claim 60 and further comprising the step of optimally balancing air to said at least one nourishment solution within said porous framework.

81. A method of tissue culturing processing according to claim 80 wherein said step of optimally balancing air to said at least one nourishment solution within said porous framework comprises the step of providing about a 50% of air and about a 50% of nourishment solution in said porous framework.

82-122. (canceled)

123. A sustentacular tissue culturing device comprising:
a plurality of explant transplant containers within which an explant growth is impacted by a punch-transplant device;
a yieldable exit element established on a bottom of said plurality of explant transplant containers;
a tissue culture growth medium contained by said plurality of explant transplant containers; and
a plurality of explants contained within said explant transplant containers and responsive to said growth medium.

124. A sustentacular tissue culturing device according to claim 123 and further comprising a synthetic retentive capability.

125. A sustentacular tissue culturing device according to claim 124 and further comprising a proper balance of said synthetic retentive capability with a plant yield ability.

126. A sustentacular tissue culturing device according to claim 123 wherein said explant transplant containers comprises a first matrix of explant transplant containers.

127. A sustentacular tissue culturing device according to claim 123 and further comprising a nourishment solution contained within said explant transplant containers.

128. A sustentacular tissue culturing device according to claim 123 wherein explant transplant containers comprises a dense population of said plurality of explants.

129. A sustentacular tissue culturing device according to claim 123 and further comprising post transplant containers in a less dense population than said explant transplant containers.

130-138. (canceled)

139. A sustentacular tissue culturing device according to claim 123 wherein said tissue culture growth medium comprises open surface multidirectional porous framework.

140. A sustentacular tissue culturing device according to claim 139 wherein said open surface multidirectional porous framework comprises open surface multidirectional porous framework capable of substantial uniform distribution of a nourishment solution.

141. A sustentacular tissue culturing device according to claim 140 wherein said open surface multidirectional porous framework capable of substantial uniform distribution of a nourishment solution comprises an open surface multidirectional porous framework capable of almost equal distribution of a nourishment solution throughout said open surface multidirectional porous framework.

142. (canceled)

143. A sustentacular tissue culturing device according to claim 139 and further comprising an ample contact between at least part of said explant and said pocket.

144. A sustentacular tissue culturing device according to claim 143 wherein said ample contact between at least part of said explant and said pocket comprises a percentage contact value selected from the group consisting of:
greater than about 25%;
greater than about 30%; and
greater than about 35%.

145. (canceled)

146. A sustentacular tissue culturing device according to claim 139 and further comprising a nourishment solution distributor and an affirmative nourishment solution eliminator.

147. A sustentacular tissue culturing device according to claim 146 wherein said open surface multidirectional porous framework comprises a nourishment solution exchange capacity and nourishment solution removal capacity balance element within said open surface multidirectional porous framework.

148. A sustentacular tissue culturing device according to claim 147 wherein said affirmative nourishment solution eliminator comprises a removal pressure of a nourishment solution greater than a retentive force said nourishment solution.

149. A sustentacular tissue culturing device according to claim 146 wherein said affirmative nourishment solution eliminator comprises a substantial nourishment solution remover element.

150. A sustentacular tissue culturing device according to claim 146 wherein said nourishment solution distributor comprises a distributor selected from the group consisting of a first nourishment solution distributor, a second nourishment solution distributor, and a refresher nourishment solution distributor.

151. A sustentacular tissue culturing device according to claim 139 and further comprising a plurality of substantially uniform interstitial voids defined by said open surface multidirectional porous framework.

152. A sustentacular tissue culturing device according to claim 151 wherein said plurality of substantially uniform interstitial voids comprises a size difference of less than about 25%.

153. A sustentacular tissue culturing device according to claim 151 wherein said plurality of substantially uniform interstitial voids comprises at least some large and at least some small voids.

154. A sustentacular tissue culturing device according to claim 153 wherein said at least some large and at least some small voids comprises a ratio of said large to small voids selected from the group consisting of:
about 3 to about 40; and
about 5 to about 40.

155. (canceled)

156. A sustentacular tissue culturing device according to claim 139 and further comprising an undistorted growth transport field of said open surface multidirectional porous framework.

157. (canceled)

158. A sustentacular tissue culturing device according to claim 139 and further comprising an optimal balance of air and a nourishment solution within said open surface multidirectional porous framework.

159. A sustentacular tissue culturing device according to claim 158 wherein said an optimal balance of air and a nourishment solution within said open surface multidirectional porous framework comprises a comprises about a 50% of air and about a 50% of nourishment solution.

160-167. (canceled)
